(19) United States
(12) Patent Application Publication
(10) Pub. No.: US 2007/0238962 A1
(43) Pub. Date: Oct. 11, 2007

(54) TRANSFER OF TREATMENT PLANNING INFORMATION USING STANDARD IMAGE TRANSFER PROTOCOLS

(76) Inventors: Andreas Hartlep, Weyarn (DE); Nits Frielinghaus, Heimstetten (DE)

Correspondence Address: DON W. BULSON (BrainLAB), RENNER, OTTO, BOISSELLE & SKLAR, LLP, 1621 EUCLID AVENUE - 19TH FLOOR, CLEVELAND, OH, 44115, US

(21) Appl. No.: 11/697,310

(22) Filed: Apr. 6, 2007

Related U.S. Application Data
(60) Provisional application No. 60/745,031, filed on Apr. 18, 2006.

(30) Foreign Application Priority Data
Apr. 6, 2006 (EP) 06007269

(51) Int. Cl. A61B 5/05

(52) U.S. Cl. 600/407

(57) ABSTRACT

A system and method for creating, generating, saving and/or exporting medical data is provided. The medical data includes image data, and the medical data conforms to an external standard, wherein additional data, not part of the external standard, is added to the image data. The additional data may be added by rendering the additional data into the image data to create new data including at least part of the original image data and at least a part of the additional data, wherein the new data conforms to the external standard.

### RELATED APPLICATION DATA

This application claims priority of U.S. Provisional Application No. 60/745,031 filed on Apr. 18, 2006, which is incorporated herein by reference in its entirety.

### FIELD OF THE INVENTION

The present invention relates to a method and a device for generating, exporting and/or saving data, such as medical image data. More particularly, the invention relates to including non-standard data (e.g., additional image information or data not part of the standard) into a data structure that conforms to a particular standard, such as DICOM.

### BACKGROUND OF THE INVENTION

Since medical imaging equipment typically interoperates with other medical devices, it is common for medical scanners (e.g., CT, MR, PET, or SPECT scanners), other medical imaging devices, displays and/or software to be interoperable so as to exchange data based on a common standard or protocol. An example of such a protocol is the DICOM (digital imaging and communication in medicine) standard published by the National Electrical Manufacturers Association. The DICOM standard includes syntax and semantics of commands and associated information that can be exchanged by devices, particularly medical imaging equipment that use the protocol. By using an interoperability standard, the exchange of digital information between medical imaging equipment can be facilitated and medical image data can be easily shared and used among compliant devices, such as imaging systems and workstations. Preferably, interfaces used to exchange such data are based on a standard, such as the DICOM standard.

A typical use of the DICOM standard is with CT or MR images. Since the image data is typically processed, post-scan converted data, once the MR or CT images are stored, none of the post-processing capabilities normally available on the MR or CT system, such as gray-scale maps, edge enhancement, and video filters, are available to enhance the CT or MR image. This provides the benefit of ensuring that the archived image reproduces as closely as possible that which the clinician who stored the image was viewing at the time the image was archived.

FIG. 4 shows in principle a data set representing medical image data according to the DICOM standard, including image data 300 and, as a header, public data 320 and private data 310. The standardized public data 320 includes information concerning the interpretation or “meaning” of the image data 300.

With reference to FIG. 3, if image data is used in devices and/or software that does not conform to the DICOM standard, additional information can be added in a header 305. This additional information can be treatment data defining, for example, trajectories or surgical pathways for planning the treatment of the tissue, or any other kind of data.

A clinician also may desire further information, e.g., further image data for planning a treatment, be included with or added to the DICOM data. Such further image data may be helpful for the further use of the DICOM data, e.g., for surgical planning. The DICOM standard, however, does not allow the inclusion of additional image data such as treatment data (e.g., trajectory information or other treatment data).

US 2005/0074157 A1 discloses a method for displaying or manipulating medical image data, wherein a file in compliance with a medical image standard is provided to a medical image viewer. The medical image standard specifies a first field for data not in compliance with the medical image standard and a second field for data in compliance with the medical image standard, wherein the first field of the file comprises medical image data and the second field of the file comprises information that can be used to obtain software to at least display or manipulate the medical image data. Using the obtained software, the medical image data can be displayed or manipulated.

### SUMMARY OF THE INVENTION

A method is provided for creating, generating and/or exporting medical data, including treatment planning data such as surgical planning data, wherein the data conforms to an external standard (e.g., the DICOM standard). The medical data includes the addition of information (e.g., additional image data) that is not part of the of the external standard. This additional information or “data”, however, is added to the medical data so as to conform to the external standard. For example, the non-standard additional data may be included with standard data by rendering the additional data (e.g., as treatment planning information) directly into the standard data (which conforms to the external standard). This creates a new data set comprising at least a part of the additional data (which originally did not conform to the external standard) and at least part of the original medical data (which conformed to the external standard) in a format that conforms to the external standard. Therefore, the new data set can be exported for use by other devices.

Using the above method, additional data (e.g, medical treatment planning information such as surgical access paths or trajectories) not normally supported by the DICOM standard can be represented in the DICOM standard. Thus, surgical planning data that includes trajectory information can be made to conform to an external standard and be exported to other devices without changing the external standard or using non-public and, thus, generally unsupported header entries.

In other words, the image data 300 itself of the DICOM data is modified by altering or replacing parts of the image data 300, e.g., specific pixels, to add or draw additional image data 330. The additional data represents, for example, trajectories, wherein the data is placed directly into the image data 300 without modifying the header 310, 320 of the DICOM data.

Exporting data conformal to a standard (e.g., DICOM) that is supported by a variety of systems, wherein the data includes data such as planning information 330 directly rendered into the image data 300 provides the following advantages. Internal data of a medical system can be made accessible to “foreign systems” without disclosing internal standards, even if the common standard (e.g., DICOM) does not allow the addition of graphic objects. Further, the external standard need not to be changed or adapted and thus, full compatibility is provided.

Thus, the method enables the transfer of medical data, e.g., imaging or treatment planning data such as trajectories, using standard image transfer protocols such as DICOM. This transfer can be performed by loading image data that is in spatial relation to the treatment planning information and rendering the treatment planning information into the images, thus replacing, changing or manipulating the original image data at those points where the treatment planning information is represented or located such that the treatment planning information is visible in the data. The changes and manipulations may include changes of the (image) information and may include additions to the (image) information, e.g., objects that may have been rendered into the data set. The resulting images carrying the medical treatment planning information can be transferred using a standard image transfer protocol like DICOM and can be imported into any kind of application that is compatible with this protocol.

Patient data may be displayed using a system-internal imaging data standard, and the image data set can be used to plan trajectories of instruments or catheters for surgical treatments. To make the plans (image data and the trajectory) accessible to systems that do not support the implemented data standard, all objects (e.g., trajectories) are rendered directly into the image data set and this newly created data set is stored conformal to a standard (e.g. DICOM) that is supported by the other imaging viewing or processing system.

The embodiments described below relate to methods and systems for displaying and/or manipulating medical image data. In one embodiment, a medical image viewer in compliance with a medical image standard is provided, and a file in compliance with the medical image standard is provided to the medical image viewer. The medical image standard specifies in addition to image data a first field for data not being standardized by the medical image standard, e.g., the “private” field 310 in FIG. 4, and a second field for data being standardized by the medical image standard, e.g., the “public” field 320 in FIG. 4.

### BRIEF DESCRIPTION OF THE DRAWINGS

The forgoing and other features of the invention are hereinafter discussed with reference to the drawings.

FIG. 1 is a block diagram of an exemplary medical diagnostic CT imaging system in accordance with the invention.

FIG. 2 is a block diagram of an exemplary medical image viewer in accordance with the invention.

FIG. 3 is a simple illustration of an exemplary user specific medical image standard.

FIG. 4 is a simple illustration of the DICOM standard.

### DETAILED DESCRIPTION

The embodiments described below relate generally to diagnostic medical images. Although any type of medical image can be used, these embodiments will be illustrated in conjunction with CT images. As noted in more detail below, other types of medical images can be used, and the following claims should not be limited to CT images unless explicitly recited therein.

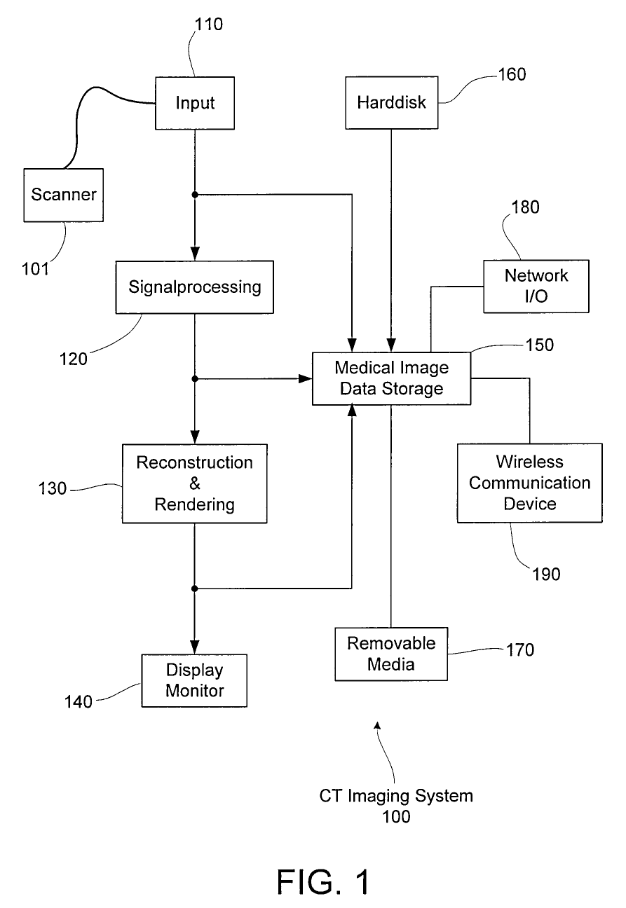

FIG. 1 is a block diagram of an exemplary CT imaging system 100. The CT imaging system 100 includes a CT scan data input port 110 connected to a CT scanner 101, a signal processing section 120, a reconstruction and rendering section 130, and a display monitor 140. The CT system 100 also includes a medical image data storage section 150, which can capture and/or store image data at one or more locations along the image path, a hard disk 160, removable media 170 (e.g., a CD, a DVD, etc.), a network I/O port 180, and a wireless communication device 190.

During a CT examination, the CT scanner 101 produces image data referred to as “CT data” and sends this image data via the input port 110 to the signal processing section 120. The signal processing section 120 evaluates the CT data and transmits it, e.g., as DICOM data, directly to storage section 150, or to the reconstruction and rendering section 130 for data modification. The reconstruction and rendering section 130 can provide the rendered data to the display 140 and/or storage section 150.

For example, if it is desired export data that conforms to a particular standard along with data that does not conform to the standard, then both the conforming and non-conforming data are provided to the reconstruction and rendering section 130. The reconstruction and rendering section 130 then renders the non-conforming data (e.g., non-conforming image data) into the conforming data (e.g., conforming image data) so as to create new image data that includes both the conforming data and the non-conforming data in a single package or group. Rendering of the data may include, for example, altering data, such as pixels, of the conforming data so as to include or otherwise represent the non-conforming data within the conforming data. Once the new image data has been rendered, the data is reconstructed into the utilized standard, thereby providing a new data set that conforms to the standard and, thus, may be exported to other devices.

The medical image data storage section 150 captures image data that is DICOM compliant. The captured data can comprise 2D images, 3D data or rendered 2D or 3D data. The image data output by the signal processing section 120 could be 2D image data or 3D image data before scan conversion, rendering or reconstruction. This image data may be readable by DICOM-compliant devices such as DICOM workstations. The captured image data also can be stored on the hard disk 160 and/or removable media 170. The captured image data also may be exported from the CT system 100 via the network I/O 180 (e.g., across an intranet or the Internet) or via the wireless communication device 190.

The CT system 100 operates in compliance with a medical image standard and sends captured medical image data to another device operating in compliance with the medical image standard. Although any medical image standard now existing or developed in the future can be used, the DICOM standard will be used to illustrate this embodiment.

In operation, the medical image data storage section 150 packages the medical image data in a file that is sent to a medical image viewer via removable media 170, the network I/O 180, or the wireless communication device 190. As used herein, the term “medical image viewer” broadly refers to any device that can be used to view and/or manipulate medical image data. Examples of medical image viewers include, but are not limited to, dedicated workstation (e.g., image review stations), general-purpose computers, personal digital assistants, cell phones, and set-top boxes. A medical image viewer also can be a medical imaging system (e.g., a CT system) different from the one used to generate the medical image data.

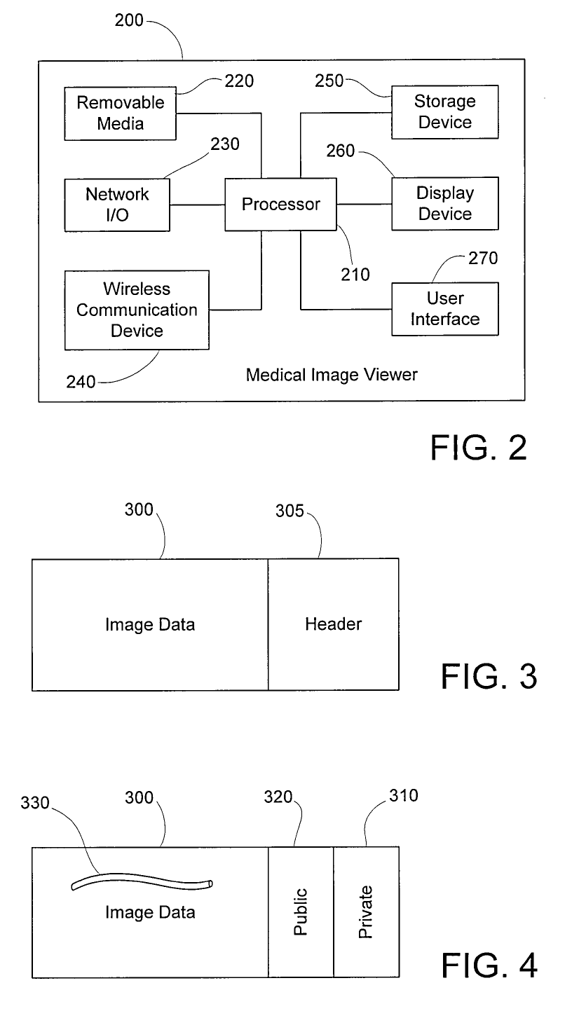

FIG. 2 is a block diagram of an exemplary medical image viewer 200 that may be used to view data from the CT scanner 100. As shown in FIG. 2, the medical image viewer 200 includes a processor 210 in communication with removable media 220, a network I/O 230, and a wireless communication device 240 that interfaces with the CT system's removable media 170, network I/O 180, and the wireless communication device 190, respectively. The medical image viewer 200 also includes a storage device 250 that can store the transferred medical image data file (and/or computer-readable program code executable by the processor 210), a display device 260, and a user interface 270.

Since the CT system 100 and medical image viewer 200 operate in compliance with the same medical image standard, medical image data that is in compliance with the medical image standard can be viewed by the medical image viewer 200 (i.e., the processor 210 of the medical image viewer 200 runs medical-image-standard-compliant viewing software). The medical image viewer 200 also can display and/or manipulate medical image data that is not in compliance with the medical image standard (e.g., pre-scan converted data, pre-reconstruction data, and a three-dimensional data set). Thus, the feature of incorporating additional image data and/or manipulating medical images into the image data file sent to the medical image viewer 200 is provided. This will be discussed in more detail with reference to FIG. 4.

As shown in FIG. 4, the medical image standard specifies that a file associated with medical image data has, in addition to the medical image data 300, a first field 310 and a second field 320 (the medical image standard can also specify additional fields, that, for simplicity, are not shown in FIG. 4). The first field 310 is for data that is not standardized in compliance with the medical image standard, and the second field 320 is for data that is standardized in compliance with the medical image standard. In the DICOM medical image standard, the first field 310 is the DICOM private attribute, and the second field 320 is the DICOM standard or public attribute.

One example of an additional image data is data showing trajectories 330 for a planned insertion of a catheter into the tissue to be shown in the image data 300. This additional image data 330 is rendered directly into the original image data 300, e.g., by modifying or replacing the pixels at the location of the planned trajectories without modifying any of the private or public fields 310 or 320. The advantage of this approach is that it does not require any new or special capability from the DICOM workstation.

It is noted that this modification of the original image data 300 can be made either in the CT system 100 shown in FIG. 1, in the image viewer 200 shown in FIG. 2 or at any other location or device.

In summary, the exemplary implementation described herein can enable a user to store medical image data 300 that is in an industry standard format (such as DICOM) including additional information 330 that is not part of the original medical image.

Although the invention has been shown and described with respect to a certain preferred embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

What is claimed is:

1. A method for creating, generating, saving and/or exporting medical data that includes image data, said medical data conforming to an external standard, wherein additional data, not part of the external standard, is added to the image data, comprising rendering the additional data into the image data to create new data including at least part of the original image data and at least a part of the additional data, wherein the new data conforms to the external standard.

2. The method according to claim 1, wherein rendering includes reconstructing the rendered data to conform to the external standard.

3. The method according to claim 1, wherein the additional data is treatment planning information.

4. The method according to claim 3, wherein the treatment planning information is at least one of trajectory information, surgical pathways, or labels.

5. The method according to claim 1, wherein the external standard is the DICOM standard.

6. The method according to claim 1, wherein rendering the additional data into the image data includes at least one of changing the image data or adding the additional data to the image data.

7. A medical image generating device, comprising:
an imaging device;
a processing device operatively coupled to said imaging device; and
logic executable by said processor, said logic including
logic that obtains image data from said imaging device, wherein the image data conforms to an external standard, and
logic that renders additional image data into the image data obtained from the imaging device to generate new data including at least part of the additional image data, the new data conforming to the external standard.

8. The device according to claim 7, wherein the logic that renders additional image data includes logic that reconstructs the rendered data to conform to the external standard.

9. The device according to claim 7, wherein the additional data is treatment planning information.

10. The device according to claim 9, wherein the treatment planning information includes at least one of trajectory information, surgical pathways, or labels.

11. The device according to claim 7, wherein the external standard is the DICOM standard.

12. The device according to claim 7, wherein the logic that renders the additional data into the image data includes at least one of logic that changes the image data or logic that adds the additional data to the image data.

13. The medical image generating device according to claim 7, wherein the imaging device is at least one of a PACS system, CT scanner, MR scanner, PET scanner, or SPECT scanner.

14. A computer program embodied on a computer readable medium for creating, generating, saving and/or exporting medical data that includes image data, said medical data conforming to an external standard, wherein additional data, not part of the external standard, is added to the image data, comprising code that renders the additional data into the image data to create new data including at least part of the original image data and at least a part of the additional data, wherein the new data conforms to the external standard.
